(19) United States
(12) Patent Application Publication
(10) Pub. No.: US 2025/0359733 A1
(43) Pub. Date: Nov. 27, 2025

(54) MEDICAL INSTRUMENT AND MEDICAL INSTRUMENT MANUFACTURE

(71) Applicant: Karl Stroz SE & Co. KG, Tuttlingen (DE)

(72) Inventors: Marvin KÄCHELE, Tuttlingen (DE); Rainer HERMLE, Tuttlingen (DE); Wolfgang KARL, Tuttlingen (DE)

(21) Appl. No.: 19/212,878

(22) Filed: May 20, 2025

(30) Foreign Application Priority Data
May 21, 2024 (DE) 10 2024 114 070.4

(51) Int. Cl. A61B 1/00; B23K 26/38

(52) U.S. Cl. CPC A61B 1/00135; A61B 1/00098; A61B 1/0011; B23K 26/38

(57) ABSTRACT

A medical instrument (30, 230) has a shaft (34) extending between a distal end (38) and a proximal end (40). A housing (36) is provided at the proximal end (40) of the shaft (34). At least one effector (60) is provided at the distal end (38) of the shaft (34). The instrument (30, 230) has at least one control element (72) for mechanically controlling the effector (60). The control element (72) extends at least in portions through a guide tube (70) extending parallel to the shaft (34). The guide tube (70) is provided at least in portions along its longitudinal extent (104) with lateral cutouts (100), through which the guide tube (70) is accessible for cleaning media. A method is used for manufacturing a guide tube (70) of a medical instrument (30, 230).

### CROSS REFERENCE TO RELATED APPLICATIONS

This application claims the benefit of priority under 35 U.S.C. § 119 of German Application 10 2024 114 070.4, filed May 21, 2024, the entire contents of which are incorporated herein by reference.

### TECHNICAL FIELD

The present disclosure relates to a medical instrument, in particular to an instrument in the form of a working insert for a medical system. Medical systems in the form of multi-part instruments are known; by way of example, they are cystoscopes (compare also cysto-urethroscopes), hysteroscopes, resectoscopes, or the like. In general, the present disclosure relates to medical instruments for diagnostic, therapeutic, and/or surgical procedures in a cavity in the body of a patient.

### BACKGROUND

Such Cystoscopes can basically be designed as flexible cystoscopes or as rigid cystoscopes. Depending on the specific application, different (individual) instruments can be combined with one another in the case of multi-part instruments. By way of example, a cystoscopic system comprises a rigid outer shaft (cystoscope shaft), which is designed to receive an insert, which serves as a working insert and/or observation insert. The insert can in turn be used to receive an observation instrument (endoscope) and/or to receive other instruments (flexible or rigid instruments such as forceps, grippers, scissors, electrosurgical instruments, and the like).

Furthermore, so-called working inserts that allow deflection of flexible instruments at their distal end are known. For example, a so-called Albarran lever, by way of example, which is mechanically coupled to a handle (wheel, lever, or the like) on a proximal housing via control elements (control cables or the like), is used for this purpose. In this way, the Albarran lever can be actuated in order to deflect an instrument at the distal end laterally relative to the (global) longitudinal extent of the working insert. In other words, an Albarran lever can be used to control the orientation/deflection of a catheter at a tip of a cystoscope. Albarran levers and similar mechanisms can also be used with instruments other than cystoscopes.

A multi-part urological instrument with an Albarran elevator is known, for example, from U.S. Pat. No. 4,178,920 A. Cystoscopes with Albarran levers are also known from DE 1 997 000 U and DE 77 06 935 U1. An instrument in the form of a hysteroscope with an Albarran lever is known from DE 100 09 020 A1. Designs of Albarran inserts are also known from EP 4 205 629 A1 and EP 4 238 475 A1.

The cleaning and sterilization of instruments with Albarran levers and similar instruments is sometimes challenging because they usually have a miniaturized mechanism with a guide of the control element from proximal to distal to the deflectable lever. The guide or control element can become dirty/contaminated. Due to miniaturization, special attention must be paid to ensuring that the cleaning fluids used actually reach the potentially contaminated regions.

Seemingly obvious measures to facilitate the cleaning of such instruments often cannot be implemented simply because there is no space available due to miniaturization. The actuation of any effectors via control elements must also not be impaired, for example with regard to positioning accuracy and repeatability. The cleaning/sterilization can be done, for example, with steam and/or cleaning liquid.

There are regulations and aids for cleaning Albarran working inserts and similar instruments. However, it has been shown that the success of the cleaning also depends on the experience and commitment of the specialist responsible for the cleaning. It should also be noted that the cleaning of the instruments is partly highly automated and partly manual. In any case, the desired cleaning result should be achieved reliably and reproducibly.

### SUMMARY

Against this background, the present disclosure is based on the object of specifying a medical instrument that is optimized with regard to cleaning and sterilization. In particular, neuralgic elements and portions of the instrument should be easily accessible for cleaning fluids. The improved cleanability should be achievable as far as possible without adverse effects on the functionality of the instrument. In particular, the instrument should be usable as an Albarran working insert for use with cystoscopes and similar multi-part instruments. In particular, the guide for control elements of an Albarran lever or similar effectors should be optimized with regard to cleanability. Finally, the present disclosure is intended to provide a method for manufacturing a guide tube for a medical instrument, the guide tube being suitable in particular for control elements for Albarran levers and similar effectors and therefore being intended to be configured to have as small a diameter as possible.

According to a first aspect, the present disclosure relates to a medical instrument, in particular a working insert for a cystoscope, hysteroscope or resectoscope, comprising:
a shaft extending between a distal end and a proximal end,
a housing at the proximal end of the shaft,
at least one effector at the distal end of the shaft,
at least one control element for mechanical control of the effector,
the control element extending at least in portions through a guide tube extending parallel to the shaft, and
the guide tube being provided at least in portions along its longitudinal extent with lateral cutouts, through which the guide tube is accessible for cleaning media.

The object of the disclosure is achieved in this way.

An instrument configured according to the disclosure has improved cleanability because the cutouts of the guide tube allow cleaning media to flow in and out. This allows a potentially problematic region to be reliably cleaned and sterilized.

In particular, the guide tube is a guide tube with a small diameter. A typical diameter of a shaft of a medical instrument is, for example, 4 mm, 8 mm, up to 10 mm, or even 12 mm. A guide tube according to the disclosure usually has a significantly smaller diameter, for example a diameter of 2 mm or 1 mm.

The shaft of the instrument can form a working channel. The at least one guide tube serves as a guide for a control element for controlling the effector. The effector can, by way of example, be a component of a deflection mechanism, such as a deflection lever, in particular a so-called Albarran lever.

The control element can also be called a control cable. By way of example, it may be a Bowden cable, a wire, a push-pull rod, and a similar string-shaped control element. Control elements are known that are primarily actuated by tension so that, for example, two states of the effector are actuated with two control elements. However, control elements are also known that are actuated by both tension and pressure.

For the purposes of the present disclosure, a proximal portion or region is a portion or region that is located closer to the observer/user and further away from the field of view/patient than a distal portion or region. Similarly, a distal portion or region is a portion or region that is closer to the field of view/patient and further away from the observer/user than a proximal portion or region. Accordingly, distal can also be described as close to the patient, facing the patient, and/or distant from the observer. Proximal can also be described as distant from the patient, facing away from the patient, and/or close to the observer. When used as an endoscopic instrument, the distal end of the shaft is usually inserted into the body in order to allow observations to be made there. At least the proximal end of the instrument protrudes from the body because this is where the operator handles and controls it.

The distal end is usually the distal end region of the shaft, not just an outermost tip of it. The instrument is configured, at least in exemplary embodiments, as a rigid instrument with a rigid shaft. The shaft usually defines a main extension direction of the instrument. The shaft of the instrument usually has a longitudinal extent that is many or several times the transverse extent (diameter or the like).

In exemplary embodiments, the at least one guide tube is arranged on an outer side of the shaft. In other words, according to these embodiments, the guide tube is not positioned inside the shaft.

The guide tube (and also the shaft) is usually made of metal materials, for example stainless steel, in particular surgical stainless steel. Manufacturing from other metals, such as a titanium alloy or an aluminum alloy, is also conceivable in principle.

The instrument is, by way of example, a working insert for a cystoscope. In general, the instrument is an instrument for diagnostic and therapeutic applications, examination and treatment of organs and tissues.

The lateral cutouts can be described as longitudinal slots along the longitudinal extent of the guide tube. The lateral cutouts provide a fluidic connection between the interior of the guide tube and the environment. In this way, cleaning fluids (liquid or gaseous) can flow in and out.

The housing can be configured in multiple parts. The shaft is coupled to the housing at its proximal end. By way of example, the shaft opens with its proximal end into the housing. The housing allows handling of the instrument. Furthermore, actuating elements and the like can be arranged on the housing.

According to an exemplary embodiment, the effector is a deflectable lever at the distal end of the shaft, in particular an Albarran lever. An Albarran lever can be used to deflect cannulas and other instruments at the distal end of the shaft. The degree of deflection can be sensitively controlled. Control elements for the Albarran lever extend, by way of example, through guide tubes according to the disclosure parallel to the shaft between the housing and the Albarran lever. Components of the Albarran lever controller should be as miniaturized as possible so that the medical system (e.g., cystoscope) as a whole can provide as many functions as possible with a given outer diameter.

According to a further exemplary embodiment, an actuating element for actuating the effector is formed on the housing, with which actuator the control element can be moved distally or proximally. In other words, the actuating element is connected to the effector via an effector controller arranged on or in the housing and via the at least one control element. The actuating element is, by way of example, a wheel or lever that is mounted on the housing.

According to a further exemplary embodiment, the guide tube is fastened to the shaft in a first circumferential portion, the cutouts being formed on a second circumferential portion of the guide tube facing away therefrom. In this way, the cleaning fluid can easily pass through the cutouts without being adversely affected by adjacent wall portions of the shaft. According to an exemplary embodiment, the guide tube is fastened to the outside of the shaft.

According to a further exemplary embodiment, multiple cutouts form a row along the longitudinal extent of the guide tube. By way of example, this is a (single) row of longitudinal slots along the longitudinal extent of the guide tube. In principle, it is also conceivable to introduce multiple rows of longitudinal slots into the guide tube. The cutouts arranged in a row are arranged one behind the other and spaced apart from one another. The main extension direction of the row is parallel to the longitudinal extent of the guide tube.

According to a further exemplary embodiment, the cutouts are configured as elongated holes or ellipses (elliptical holes), the cutouts having a longitudinal extent and a transverse extent, and the longitudinal extent being at least twice the transverse extent. In an exemplary embodiment, this applies to at least some or all of the cutouts. The cutouts preferably provide sufficiently large openings for the cleaning fluid without significantly reducing the structural integrity and stability of the guide tube.

According to a further exemplary embodiment, the cutouts have a longitudinal extent which is between 10 mm and 30 mm, in particular between 15 mm and 25 mm, and/or the cutouts have a transverse extent which is between 0.15 mm and 1.0 mm, in particular between 0.25 and 0.4 mm. In this way, cutouts that allow good accessibility for the cleaning fluid can be created even in miniaturized guide tubes.

According to a further exemplary embodiment, the guide tube has an outer diameter which is less than 2.2 mm, preferably less than 1.8 mm, more preferably less than 1.2 mm, and/or the guide tube having a wall thickness which is less than 0.3 mm, preferably less than 0.18 mm, more preferably less than 0.13 mm. In an exemplary embodiment, the outer diameter of the guide tube is 1.0 mm, with a wall thickness of 0.1 mm. The guide tube is usually a component based on a cylindrical tube with a constant circular cross-section. Any cutouts are created in the tube by removing material.

The shaft of the instrument also usually has a circular cross-section. However, designs of the shaft with a cross-section that differs from a circle are also conceivable.

According to a further exemplary embodiment, the cutouts are cut out with little or no post-processing by means of ultrashort pulse laser processing. This is advantageous for the wear behavior and ease of movement during operation of the instrument when the control element is moved along the longitudinal extent within the guide tube. Given the miniaturized components, post-processing the cutouts, for example in the region of their edges, would involve excessive effort.

Ultrashort pulse laser processing allows for the high-precision production of delicate structures. One advantage of ultrashort pulse laser processing is its suitability for hard materials. Furthermore, in ultrashort pulse laser processing, a high energy input occurs in a very short time due to the extremely short pulses, which ensures that the material changes directly from the solid to the gaseous state and evaporates. Surrounding material is not or only minimally thermally stressed, which makes highly precise material removal possible. This process is called sublimation.

Machining by means of an ultrashort pulse laser to create the cutouts in the guide tube allows the creation of a precise contour with highly accurate surfaces and edges, even in guide tubes with small diameters. Machining by means of an ultrashort pulse laser makes the creation of such cutouts possible even in guide tubes with a diameter of less than 2.0 mm down to a diameter of about 1.0 mm or even less. Tubes with such small diameters cannot be machined with the required precision using conventional laser machining methods or other material-removing machining methods.

According to a further exemplary embodiment, edges of the cutouts, in particular inner wall edges, are configured to be low in burrs or burr-free. This is beneficial for the ease of movement of the control element during operation. Furthermore, the control element can be operated in the guide tube with low wear.

According to a further exemplary embodiment, the cutouts have side walls, opposite side walls of a cutout enclosing an outward-opening angle that is greater than 5°, preferably greater than 15°, more preferably greater than 25°. The outward-opening angle points with its open side away from the longitudinal axis of the guide tube. According to a further exemplary embodiment, the side walls are oriented radially to a longitudinal axis of the guide tube.

When machining with an ultrashort pulse laser, the cutouts are created by cutting out sections from the wall of the guide tube. The design of the side walls inclined toward one another (for example, opposite walls on the long sides with a cutout) helps to ensure that a cut-out section separated from the surrounding material of the guide tube does not fall into the interior of the guide tube. In this way, process reliability during the production of the cutouts increases because the cut-out sections (waste pieces) can be safely removed.

According to a further exemplary embodiment, the length of the inner wall edges of a cutout is smaller than the length of the outer wall edges of the cutout. This applies to one entire circumference along the edges. This also prevents the cut-out sections from falling in. According to a further exemplary embodiment, the cutouts are tapered at least in portions in the direction of the longitudinal axis.

According to a further exemplary embodiment, a first guide tube and a second guide tube are arranged on the shaft, which guide tubes are in particular fastened to the outside of the shaft, the first guide tube accommodating a first control element and the second guide tube accommodating a second control element, and the first control element and the second control element being coupled to the effector at the distal end. In this way, the effector can be controlled symmetrically. This is advantageous, for example, if a (further) instrument is guided along the shaft between the two guide tubes. Two control elements acting on the lever at a distance from each other is advantageous, in particular if the effector comprises a lever (Albarran lever or the like).

According to a further exemplary embodiment, the shaft is connected to the housing, the shaft in particular opening into the housing, the housing providing proximal access to the shaft, and at least the shaft or the housing having at least one bore inclined relative to a shaft axis of the shaft, through which bore a housing chamber used by the controller of the effector is accessible for cleaning media.

According to this design, the cleaning fluid can flow through the shaft (and the housing) along the longitudinal extent of the shaft. The housing also serves as a support for components of an effector controller, for example a mechanism for converting a pivoting movement (of the actuating element) into a translational movement (of the control element). The bores, which are inclined relative to the shaft axis, allow a defined transfer of the cleaning fluid into the housing chamber so that the components of the instrument installed there can be cleaned.

This is advantageous, for example, if the guide tube opens into the housing chamber at its proximal end. In principle, this could lead to contamination of the housing chamber. Therefore, a defined bore in the housing that makes the housing chamber accessible is advantageous because the cleaning fluid can thus penetrate into the housing via a short route.

According to a further exemplary embodiment, the guide tube and the shaft are fluidically connected to one another via the housing chamber. In other words, the housing chamber can be flooded with the cleaning fluid when the shaft of the instrument and/or the guide tube are flooded with the cleaning fluid.

According to a further exemplary embodiment, the guide tube opens into the housing chamber. According to this embodiment, the control element can extend into the housing chamber in order to be coupled there with components of the effector controller. In principle, the housing chamber can also become contaminated in this way; cleanability can be improved by a bore in the housing.

According to a further exemplary embodiment, at least one inclined bore is configured as a transverse bore through the shaft to the housing chamber, in particular as a transverse bore oriented orthogonally to the shaft axis of the shaft. Such a transverse bore through the wall of the shaft to the housing chamber provides a direct connection within the housing to the housing chamber, which connection can be used for cleaning. It is understood that multiple such transverse bores may be provided.

According to a further exemplary embodiment, at least one inclined bore is formed as a connecting bore between a housing part at the proximal end of the shaft and the housing chamber, in particular at an acute angle to the longitudinal axis of the shaft, which angle opens distally.

The housing usually provides a seat for the proximal end of the shaft. The inclined bore can extend through this seat in the direction of the housing chamber without penetrating the shaft itself. Multiple such bores can be distributed around the shaft and can provide access to the housing chamber. For example, this is an inclined bore that extends from a proximal opening of the housing to the housing chamber. The opening of the inclined bore in the housing is, for example, in a portion which is offset, on the one hand, proximally relative to the proximal end of the shaft and, on the other hand, distally relative to a distal end of a cleaning adapter which is inserted into a proximal receptacle in the housing.

According to a further exemplary embodiment, the control element is coupled at its proximal end to a rod which extends through a wall piece into the housing, a proximal end of the guide tube being spaced from the housing. This design has the advantage that neither the guide tube nor the control element opens into the housing chamber or extends into the housing chamber.

According to a further aspect, the present disclosure relates to a method for manufacturing a guide tube of a medical instrument, in particular an instrument according to at least one of the embodiments described herein, comprising the following steps:
providing a workpiece in the form of a semi-finished product comprising a thin-walled tubular body with a longitudinal axis extending between a distal end and a proximal end,
providing an ultrashort pulse laser,
clamping the workpiece in a workpiece holder,
operating the ultrashort pulse laser to process the workpiece by means of sublimation,
creating a plurality of cutouts in the workpiece, comprising:
   generating a coordinated relative movement between the tubular body and the ultrashort pulse laser with the ultrashort pulse laser activated, comprising a translational movement along the longitudinal axis and a pivoting movement with respect to the longitudinal axis in order to create a cut-out section in the workpiece, which cut-out section defines a cutout, and
   generating a feed movement between the tubular body and the ultrashort pulse laser with the ultrashort pulse laser deactivated, comprising a translational movement along the longitudinal axis in order to create multiple cutouts that are spaced apart from one another.

In this way, too, the object of the disclosure is achieved.

By machining with an ultrashort pulse laser, particularly delicate cutouts can be created even in miniaturized workpieces. This can be done with little or no post-processing. Any post-processing would be very complex given the small dimensions of the workpieces (tubes with a diameter of, for example, 2.0 mm or 1.0 mm) so that the machining with the ultrashort pulse laser avoids additional work. The ultrashort pulse laser sublimes material so that, as part of the relative movement between the ultrashort pulse laser and the workpiece, cut-out sections are created in the workpiece that expose the cutouts.

For creating the cutout, no extensive material is removed. Instead, a relatively narrow cutting gap is created between the cut-out sections and the surrounding material of the tubes.

Another advantage of machining with the ultrashort pulse laser is that it only introduces relatively little heat into the workpiece. The structure of the workpiece is therefore not excessively affected.

The relative movement between the workpiece and the ultrashort pulse laser can be generated by a movement of the workpiece with respect to the laser when the laser is stationary. The relative movement between the workpiece and the ultrashort pulse laser can be generated by a movement of the laser with respect to the workpiece when the workpiece is stationary. A combined movement of the workpiece and the laser is also conceivable.

According to an exemplary embodiment, the method further comprises the following steps:
removing the cut-out sections radially outward, the cutouts having mutually inclined and opposite side walls, the angle of inclination of which points in the direction of the longitudinal axis, and/or
rotating the workpiece about the longitudinal axis until the cut-out sections can be removed with the assistance of gravity.

It is advantageous if opposite side walls of the cutouts are inclined toward each other so that the cut-out sections created cannot fall inward into the workpiece. The cutouts are generally tapered, at least in portions, in the direction of the longitudinal axis. This may include a radial orientation of the side walls. This makes it easier to remove the cut-out sections. If necessary, it is sufficient to rotate the workpiece (in a horizontal orientation) about its longitudinal axis until the cut-out sections can fall out downward.

A guide tube configured and/or manufactured according to the disclosure can be used to guide a control element for a medical instrument, for example for a working insert with a deflectable effector at the distal end. The guide tube can be connected to a shaft of the instrument in order to provide a guide for a control element coupled to the effector. The guide tube can be arranged within the shaft. The guide tube can be located outside the shaft. Usually, the guide tube extends parallel to the (rigid) shaft.

When cleaning such an instrument, the cutouts allow entry/exit of the cleaning fluid for cleaning the control element and components coupled thereto.

It is understood that the features mentioned above and those to be explained below can be used not only in the combination specified in each case, but also in other combinations or on their own, without departing from the scope of the present invention.

Further features and advantages of the invention will become apparent from the following description and explanation of multiple exemplary embodiments with reference to the drawings.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a medical system in the form of a multi-part instrument, with components separated from one another;

FIG. 2 is a partial perspective view of an instrument with a deflectable lever;

FIG. 3 is a partial perspective view of a guide tube of the instrument according to FIG. 2;

FIG. 4 is a sectional view illustrating a cross-section of the guide tube according to FIG. 3;

FIG. 5 is a partial side view of the guide tube according to FIG. 4;

FIG. 6 is a perspective, sectional partial view of an instrument at a proximal end of a shaft, which opens into a housing;

FIG. 7 is a perspective, partial sectional view of a further instrument at a proximal end of a shaft, which opens into a housing;

FIG. 8 is a schematic view of a manufacturing device for machining a workpiece for producing a guide tube provided with cutouts;

FIG. 9 is a perspective schematic view of the workpiece according to FIG. 8, illustrating a relative movement during machining;

FIG. 10 is a schematic side view of a guide tube obtained by machining with cut-out sections, which expose cutouts; and

FIG. 11 is a simplified block diagram illustrating an exemplary embodiment of a method for manufacturing a guide tube of a medical instrument.

### DESCRIPTION OF PREFERRED EMBODIMENTS

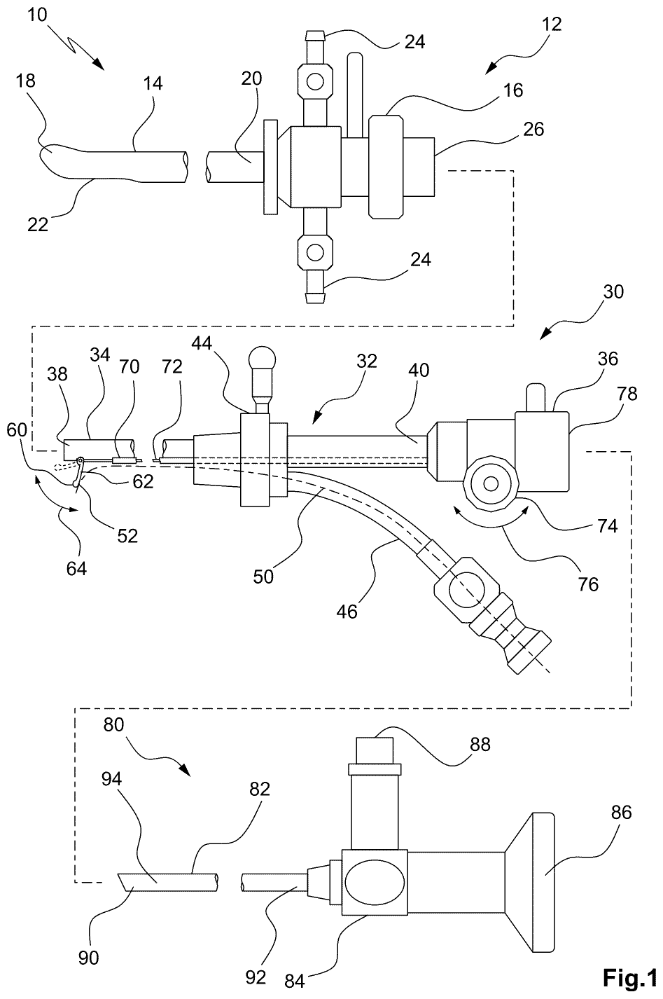

Referring to the drawings, FIG. 1 illustrates a schematic view of a medical system, designated overall by 10, in a state in which its components are not joined together. The medical system 10 can also be referred to as a multi-part instrument since it is composed of various components, which in turn can be referred to as an instrument. The system 10 is, for example, configured as a cystoscope.

The system 10 comprises a shaft part 12, which is configured, for example, as a cystoscope shaft. The shaft part 12 comprises an outer shaft 14 and a housing 16. The outer shaft extends between a distal end 18 and a proximal end 20. At the distal end 18, an opening 22 is introduced into the outer shaft 14. In the exemplary embodiment, flushing connections 24 for the passage and control of a flushing fluid are formed on the housing 16 of the shaft part 12. The housing 16 has a proximal receptacle 26, into which a further instrument 30 can be inserted into the shaft part 12.

By way of example, the instrument 30 is a working insert 32. The working insert 32 comprises a shaft 34 and a housing 36. The shaft 34 extends between a distal end 38 and a proximal end 40. The housing 36 is located at the proximal end 40 of the shaft 34. A bridge 44 is located along the longitudinal extent of the shaft 34 between the proximal end 40 and the distal end 38. The instrument 30 can be inserted with the shaft 34 into the receptacle 26 in the housing 16 of the shaft part 12. The bridge 44 can be coupled to the receptacle 26.

The instrument 30 further comprises a working channel 46 coupled to the bridge 44. A flexible instrument 50 (indicated by a dotdashed line in FIG. 1) can be inserted via the working channel 46. Such a flexible instrument 50 can then be arranged together with the shaft 34 in the outer shaft 14. The flexible instrument 50 can protrude from the outer shaft 14 at the opening 22.

The flexible instrument 50 has a distal end 52, which can be deflected by an effector 60 configured here as a lever 62. Compare also the curved double arrow 64. The lever 62 is configured, by way of example, as a so-called Albarran lever. The lever 62 is displaceable between two positions relative to the shaft 34, compare the dashed representation in FIG. 1. In this way, the distal end 52 of the flexible instrument 50 can be nestled against the shaft 34 in a first position and deflected relative to the shaft 34 in a second position.

The effector 60 can also be configured differently; it does not have to be a lever 62. The effector 60 can be controlled via a control element 72 guided in a guide tube 70. In the exemplary embodiment, the guide tube 70 extends from the distal end 38 (distal end region) to the proximal end 40 of the shaft 34. Likewise, the control element 72 extends between the distal end 38 and the proximal end 40 of the shaft 34. At the proximal end 40 of the shaft 34, the control element 72 is coupled to an actuating element 74. In the exemplary embodiment according to FIG. 1, the actuating element 74 is configured as a wheel, but a design as a lever is also conceivable. By moving the actuating element 74 (compare the curved double arrow 76), the control element 72 can be moved in order to actuate the effector 60 (compare the curved double arrow 64).

At the proximal end of the housing 36 of the instrument 30, a receptacle 78 is formed, into which a further instrument can be inserted, in the exemplary embodiment an endoscope 80.

The endoscope 80 has an endoscope shaft 82 and a housing 84. The housing 84 proximally supports an eyepiece 86 which, during operation, provides an optical image perceptible to the human eye. It is understood that in alternative embodiments, endoscopes with a camera attachment can also be used instead of the eyepiece 86. Finally, endoscopes in which the image acquisition is carried out by an image sensor, for example by a distal image sensor, without an optical image being provided in the housing 84 are also known. The endoscope 80 according to FIG. 1 further has a light connection 88 on the housing 84. By way of example, a light-conducting cable can be connected to the light connector 88 in order to illuminate the field of view. Other lighting technologies are conceivable in principle, for example lighting through integrated light sources.

The endoscope shaft 82 extends between a distal end 90 and a proximal end 92 and accommodates an observation optic 94. The endoscope 80 is configured, by way of example, as an instrument with an inclined viewing direction. However, endoscopes with a straight-forward view are also known. A conventionally configured endoscope 80 has an observation beam path and an illumination beam path in the endoscope shaft 82.

The instruments shown in FIG. 1 (shaft part 12, working insert 32, endoscope 80 and, optionally, flexible instrument 50) can be combined with one another in order to form a multi-part instrument (medical system 10). In this way, for example, a cystoscope, hysteroscope, and/or resectoscope can be provided. The possibility of deflection with the effector 60 increases the range of applications for such a multi-part instrument. However, this requires the integration of at least one control element 72 extending through a guide tube 70 from distal to proximal. The control element 72 and the guide tube 70 are installed in a region where the required installation space is important. In order to minimize the overall stress on the patient, the system 10 should have the smallest possible outer diameter in the region of the outer shaft 14. This goal can only be achieved if the components to be inserted into the outer shaft 14 also have very small cross-sections. However, this in turn leads to challenges in cleaning such components.

By way of example, cleaning the instrument 30 configured as a working insert 32 is complex because the mechanism for controlling the effector 60 may also be contaminated. The guide tube 70 with the control element 72 guided therein should therefore also be able to be cleaned in a process-safe manner.

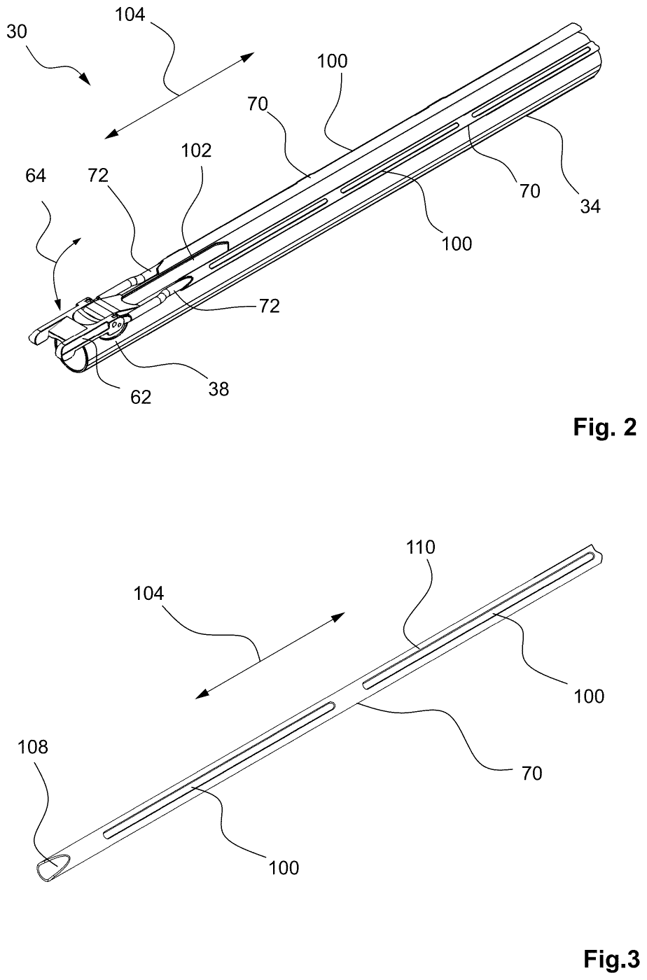

With reference to FIGS. 2 and 3, an exemplary embodiment of the instrument 30 is illustrated by means of partial perspective views. In addition, reference is made to the design according to FIG. 1, corresponding reference numbers being used for corresponding parts. The instrument 30 according to FIG. 2 can basically be used as a working insert 32 (compare FIG. 1). FIG. 2 shows the distal end 38 of the shaft 34 and an adjoining region. The deflectable lever 62 (Albarran lever) is mounted at the distal end 38; compare the curved double arrow 64 illustrating the deflection movement.

Two control elements 72, each guided in a guide tube 70, are used to control the lever 62. In the exemplary embodiment according to FIG. 2, two guide tubes 70 are accordingly fastened to the outer side of the shaft 34. The guide tubes 70 extend parallel to the shaft 34. In other words, in the exemplary embodiment, the shaft 34 and the guide tube 70 have a mutually parallel longitudinal extent (compare the double arrow 104). The guide tubes 70 are each provided along their longitudinal extent 104 with cutouts 100 through which the interior of the guide tube 70 is accessible. The shaft 34 is also open at its distal end. Furthermore, an opening 102 is formed in the shaft 34 at the circumference of the shaft 34 at the deflectable lever 62.

FIG. 3 shows an enlarged view of a guide tube 70 based on the representation in FIG. 2. The guide tube 70 is provided along its longitudinal extent 104 with multiple cutouts 100, which form a row 110. The guide tube 70 further has a distal opening 108, through which the control element 72 (compare FIG. 2) protrudes from the guide tube 70. In the exemplary embodiment, the cutouts 100 are configured as elongated holes. Cleaning fluid can flow into or out of the guide tube 70 through the cutouts 100. In other words, the control element 72 can thus also be cleaned well in the region of its longitudinal extent (arrow 104) in which it is arranged within the guide tube 70.

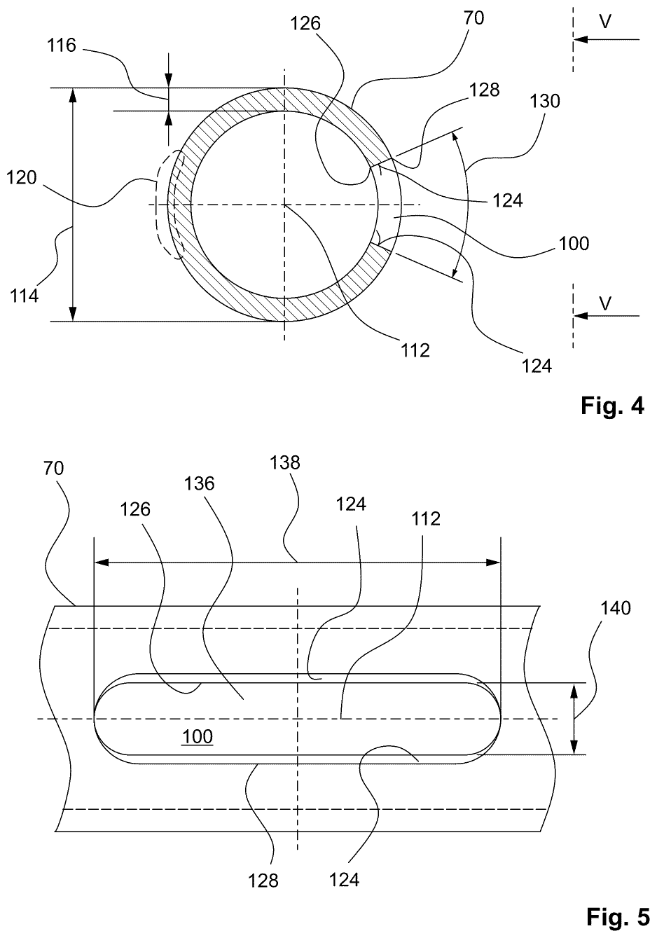

With additional reference to FIGS. 4 and 5, conceivable designs and dimensions of the guide tube 70 and of the cutouts 100 are illustrated. FIG. 4 shows a cross-section through the guide tube 70 in the region of a cutout 100. FIG. 5 illustrates a partial side view of the guide tube 70 with a front view of a cutout 100 configured as an elongated hole. The view orientation of FIG. 5 results from the line V-V in FIG. 4. A longitudinal axis 112 through the guide tube 70 is parallel to the viewing plane in FIG. 5. In FIG. 4, the longitudinal axis 112 is perpendicular to the viewing plane.

The guide tube 70 has an outer diameter 114 and a wall thickness 116. For example, the outer diameter 114 is 1.0 mm. For example, the wall thickness 116 is 0.1 mm. Other values are conceivable in principle, but the guide tube 70 should be as small as possible in order to take up as little space as possible in the medical system 10. In the case of such a miniaturized guide tube 70, the cutouts 100 cannot be produced or can only be produced with very high effort using established manufacturing techniques. A manufacturing technology suitable for producing the cutouts 100 in delicate guide tubes 72 is so-called ultrashort-pulse lasering.

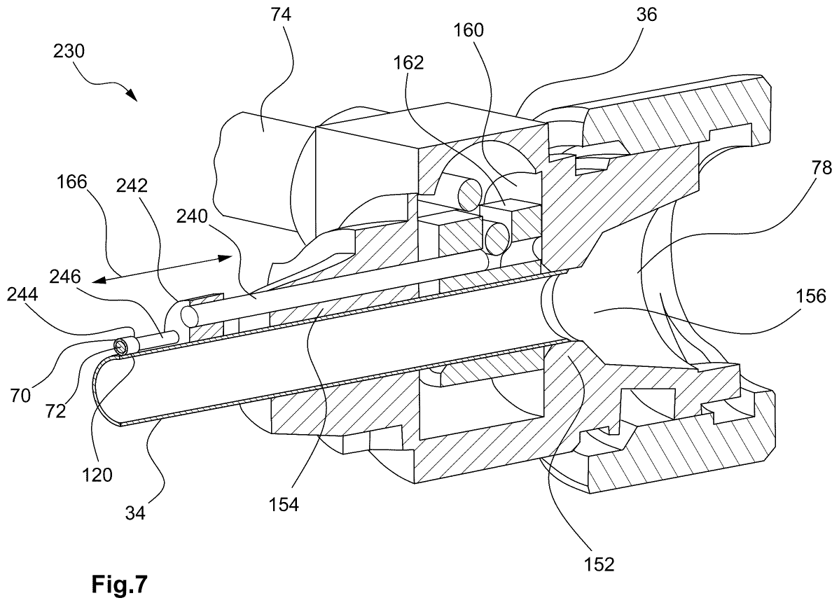

The cutout 100 is arranged in a circumferential portion on the guide tube 70, which portion is opposite a circumferential portion 120 in which the guide tube 70 is connected to the shaft 34, compare also FIG. 2 and FIG. 7.

The cutout 100 shown in section in FIG. 4 has side walls 124. Furthermore, edges (inner edges) 126 and edges (outer edges) 128 are provided. In an exemplary embodiment, the side walls 124 are oriented radially to the longitudinal axis 112. In other words, the two opposite side walls 124 shown in the representation according to FIG. 4 are not parallel to each other but are inclined in a V-shape relative to each other. An opening angle is indicated in FIG. 4 with 130. By means of ultrashort-pulse lasering, the cutouts 100 can be created with high precision. By way of example, the edges 126, 128 can be produced to be burr-free or almost burr-free. Overall, the cutouts 100 can be produced by means of ultrashort-pulse lasering without post-processing, if possible, or with as little post-processing as possible.

FIG. 5 further illustrates the design of the cutout 100 shown in section in FIG. 4. The inclination of the opposite side walls 124 in FIG. 4 results in a design in which a total length of the inner edges 126 is shorter than the total length of the outer edges 128. In other words, the cutout 100 is narrower on its inner side than on its outer side. This has the advantage that any cut-out sections that are separated from the surrounding material of the guide tube 70 during the creation of the cutout 100 cannot fall into the interior of the guide tube 70. Opposite side walls 124 of the cutout 100 do not necessarily have to be continuously inclined in a V-shape relative to each other along an entire circumference. The cutout 100 is tapered in the direction of the longitudinal axis 112.

The cutout 100 is configured as an elongated hole 136 in FIG. 5. Accordingly, the cutout 100 has a longitudinal extent 138 which is a multiple of the transverse extent 140. The longitudinal extent 138 is, for example, between 10 mm and 30 mm. The transverse extent 140 is, for example, between 0.25 mm and 0.4 mm. The representation in FIG. 5 is therefore not necessarily to scale.

The design of the cutout 100 shown in FIG. 4 and FIG. 5 can be produced, for example, if the ultrashort pulse laser is oriented perpendicularly to the longitudinal axis 112 and radially aligned with the longitudinal axis 112, a relative movement (combined linear movement and rotational movement) being generated between the guide tube 70 and the ultrashort pulse laser while maintaining the radial alignment with the longitudinal axis 112. In this way, the removal of any cut-out sections during the production of the cutouts 100 is facilitated. It is clear that contours other than the elongated hole design according to FIG. 5 are also conceivable, for example an oval design of the cutouts 100.

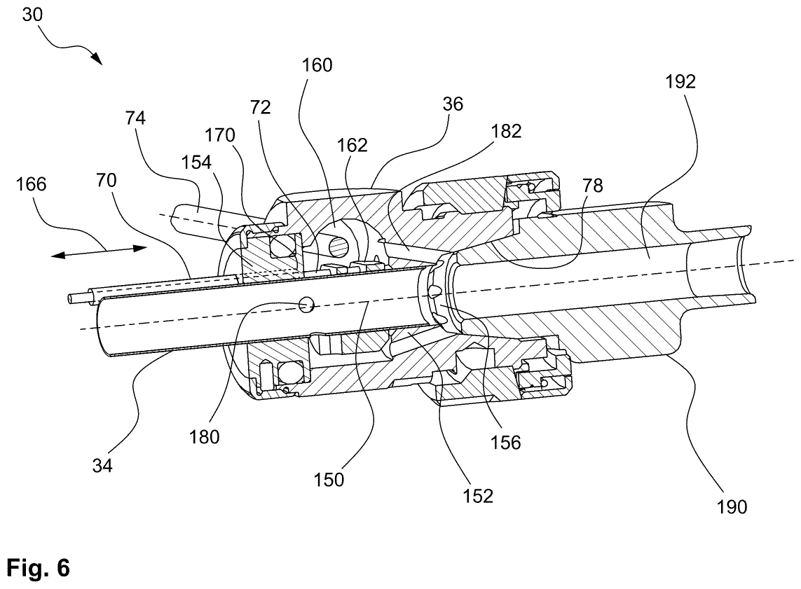

With reference to FIG. 6, an exemplary embodiment of the instrument 30 in the region of its housing 36 is illustrated by means of a perspective sectional view. In a manner already described in principle, the instrument 30 can be provided with guide tubes 70 having lateral cutouts 100 (compare FIG. 3).

The shaft 34 of the instrument 30 opens into the housing 36. A shaft axis 150 extends through the shaft 34 and describes a main extension direction for the shaft 34. The shaft 34 is a rigid shaft and is made of a suitable metal, for example surgical stainless steel. A housing part 152 is formed in the housing 36 and serves as a seat for the shaft 34. The shaft 34 extends through a wall piece 154, which is associated with the housing 36, into the housing 36. The housing 36 provides an access 156 into the shaft 34 at the proximal end of the shaft 34.

A housing chamber 160, which at least partially surrounds the shaft 34 and accommodates an effector controller 162, is formed in the housing 36. The effector controller 162 couples the actuating element 74 (compare also FIG. 1) to the control element 72 for controlling the effector 60 (compare again FIG. 1). In this way, an actuation of the actuating element 74 can be converted into a movement of the control element 72 in a proximal or distal direction, compare the double arrow 166.

In the exemplary embodiment according to FIG. 6, the control element 72 extends into the housing chamber 160. The guide tube 70, through which the control element 72 extends, extends through the wall piece 154 and opens with its proximal end 170 into the housing chamber 160. The housing chamber 160 can become contaminated via this path (movement 166 of the control element 72 in the guide tube 70). However, the penetration of a cleaning fluid is made difficult by a comparatively small gap between the control element 72 and the guide tube 70.

It is therefore conceivable to provide bores 180, 182 in the housing chamber 160, through which bores the cleaning fluid can easily penetrate into the housing chamber 162. In the exemplary embodiment according to FIG. 6, a cleaning adapter 190 is located in the receptacle 78 at the proximal end of the housing 36, compare also FIG. 1. A passage 192, through which a cleaning fluid can penetrate into the shaft 34 via the access 156, is provided via the cleaning adapter 190. In the region of the housing chamber 160, the shaft 34 has one or more inclined bores in the form of transverse bores 180, which in the exemplary embodiment are oriented orthogonally to the shaft axis 150. Alternatively or additionally, inclined bores 182 are provided through the housing part 152 serving as a seat for the shaft 34, which bores also open into the housing chamber 160. The (mouths of the) inclined bores 182 are positioned proximally in front of the shaft 34 in the region of the receptacle 78. In the exemplary embodiment, the inclined bores 182 are oriented at an acute angle to the shaft axis 150, with the resulting angle of inclination opening distally.

The bores 180, 182 can be introduced alternatively to one another or in combination in order to simplify the flooding of the housing chamber 160 with the cleaning fluid.

With reference to FIG. 7, a modified embodiment of an instrument designated by 230 is illustrated in comparison to the representation in FIG. 6. The instrument 230 is basically configured similarly to the instrument 30 and can be used as a working insert. Therefore, the following will primarily focus on features that are configured differently. Otherwise, corresponding reference numbers are used for corresponding parts. The shaft 34 opens into the housing 36, with the proximal end of the shaft 34 resting in a housing part 152 serving as a seat.

The housing 36 proximally provides a receptacle 78 (compare FIG. 1 and FIG. 6) through which an access 156 into the shaft 34 is accessible. A housing chamber 160 is formed in the housing 36, which housing chamber at least partially surrounds the shaft 34 and accommodates an effector controller 162. The exemplary embodiment of the instrument 230 according to FIG. 7 differs from the design of the instrument 30 according to FIG. 6 at least in that the control element 72 does not extend into the housing chamber 160. The guide tube 70 can be provided with cutouts 100 analogously to the previously described embodiments.

The guide tube 70 has a proximal end 244 spaced apart from the housing 36. The control element 72 also does not protrude into the housing 36 or into the housing chamber 160. Instead, a rod 240 is provided, which couples the effector controller 162 within the housing chamber 160 to the control element 72 and ultimately to the effector 60 (compare FIG. 1). In the exemplary embodiment, the rod 240 extends through the wall piece 154 into the housing chamber 160. The control element 72 protrudes with its proximal end 246 at least temporarily proximally out of the guide tube 70. The proximal end 246 of the control element 72 is coupled to a connector 242, which may also be referred to as a connecting nipple. The proximal/distal movement 166 is transmitted to the control element 72 via the connector 242. If the rod 240 is guided sufficiently tightly in the wall piece 154, there is no risk of disturbing contamination of the housing chamber 160. The control element 72 mounted in the guide tube 70 can also be cleaned in the region of its proximal end 246 outside the housing 36.

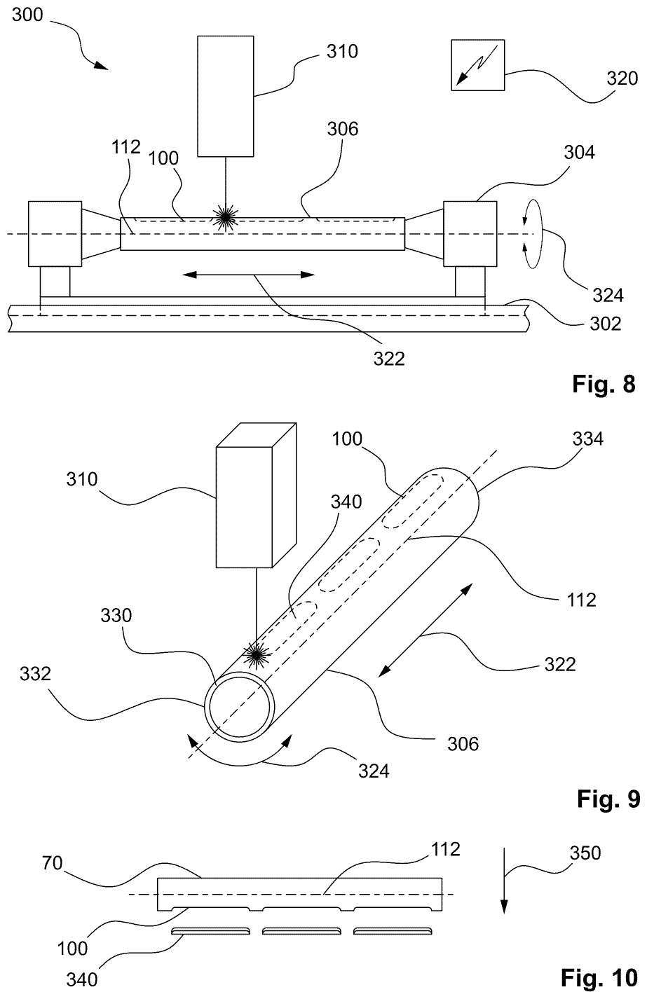

With reference to FIGS. 8 to 10, a conceivable approach for manufacturing guide tubes 70 provided with cutouts 100 is illustrated.

FIG. 8 illustrates a schematic representation of a manufacturing device designated overall by 300. In the exemplary embodiment, the manufacturing device 300 comprises a base 302, which supports a workpiece holder 304 for receiving a tubular workpiece 306. The manufacturing device 300 comprises an ultrashort pulse laser 310, which can act on the workpiece 306 in order to remove material or create cuts there.

Furthermore, a control device 320 is part of the manufacturing device 300; the control device 320 can coordinate a relative movement between the ultrashort pulse laser 310 and the workpiece 306 in order to produce the cutouts 100. The coordinated relative movement includes, for example, a translational movement 322 along the longitudinal axis 112 (for example, relative to the base 302) and a pivoting movement 324 about the longitudinal axis 312 of the workpiece 306.

FIG. 9 additionally illustrates, using a perspective schematic representation, the interaction between the ultrashort pulse laser 310 and the workpiece 306, which is configured here as a tubular body 330. With the workpiece holder 304 shown in FIG. 8, the workpiece 306 can be clamped between a proximal end 334 and a distal end 332 and rotated about its longitudinal axis 112.

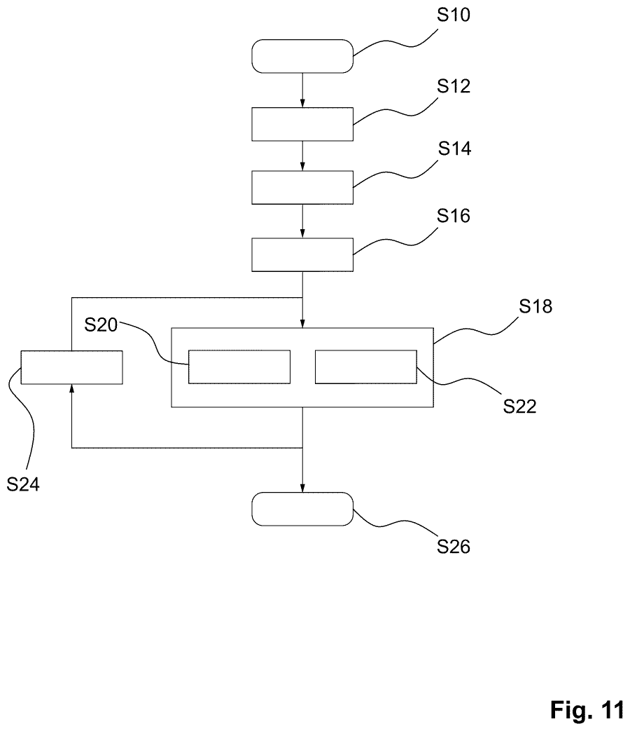

The workpiece 306 can be moved translationally (arrow 322) and rotationally (pivoting movement 324) in order to create cut-out sections 340 in the workpiece 306 with the ultrashort pulse laser 310 stationary, which cut-out sections define and expose the cutout 100 (compare FIG. 10). The cutouts 100 are configured, by way of example, analogously to the design according to FIGS. 4 and 5. This design has the advantage that the cut-out sections 340, which expose the cutouts 100, cannot fall into the interior of the workpiece 106 or of the guide tube 70 produced therefrom. FIG. 10 illustrates, using a schematically simplified representation, that the guide tube 70 can be rotated about its longitudinal axis 112 so that the cut-out sections 340 can fall out of the cutouts 100. In other words, the removal of the cut-out sections 340 can be carried out with the aid of gravity (arrow 350 in FIG. 10).

With reference to FIG. 11, an exemplary embodiment of a method for manufacturing a guide tube for a medical instrument is illustrated by means of a schematic block diagram.

In the exemplary embodiment, the method begins with a step S10 and ends with a step S26.

A step S12 relates to the provision of an ultrashort pulse laser. The ultrashort pulse laser can be part of a suitable manufacturing system for producing the guide tube. A subsequent step S14 relates to the provision of a tubular workpiece, in which multiple cutouts are to be created by means of ultrashort-pulse lasering. The workpiece is in particular a small-diameter workpiece with a small wall thickness.

In a subsequent step S16, the workpiece is received and clamped in a workpiece holder. The machining to create a cutout can then begin, compare step S18. The machining takes place with the ultrashort pulse laser activated. The machining comprises a combined relative movement, comprising a translational movement S20 and a pivoting movement S22. In this way, cutouts can be created in the tubular workpiece, which cutouts are configured, for example, as an elongated hole or oval, the respective longitudinal extent being adapted to the longitudinal extent of the workpiece. The ultrashort pulse laser can be moved along a closed path by coordinated relative movement relative to the workpiece in order to create a cut-out section.

If further cutouts are to be created, a feed movement between the workpiece and the ultrashort pulse laser is carried out in a further step S24 with the ultrashort pulse laser deactivated, so that step S18 can subsequently be carried out again in order to create a further cutout at a distance from the previously created cutout.

Steps S18 to S24 are repeated until a series of cutouts is created along the longitudinal extent of the workpiece. In this way, a guide tube with a plurality of lateral slots arranged in a row can be created.

The ultrashort pulse laser can create delicate contours in the workpiece by cutting out and removing corresponding cut-out sections. Accordingly, it is also conceivable to produce multiple rows of cutouts, cutouts within a row being offset relative to one another along the longitudinal extent, and the rows being offset relative to one another on the circumference of the workpiece. It is also conceivable in principle to combine cutouts of different contours with one another.

While specific embodiments of the invention have been shown and described in detail to illustrate the application of the principles of the invention, it will be understood that the invention may be embodied otherwise without departing from such principles.

What is claimed is:

1. A medical instrument comprising:
a shaft extending between a distal end and a proximal end;
a housing at the proximal end of the shaft;
at least one effector at the distal end of the shaft;
at least one control element for mechanically controlling the effector; and
a guide tube extending parallel to the shaft, wherein the control element extends at least in portions through the guide tube and wherein the guide tube is provided at least in portions, along a guide tube longitudinal extent, with lateral cutouts, through which the guide tube is accessible for cleaning media.

2. A medical instrument according to claim 1, wherein the effector comprises a deflectable lever at the distal end of the shaft.

3. A medical instrument according to claim 1, further comprising an actuating element for actuating the effector, wherein the actuating element is formed on the housing and the actuating element is configured to move the control element distally or proximally.

4. A medical instrument according to claim 1, wherein the guide tube is fastened to the shaft in a first circumferential portion, and wherein the cutouts are formed on a second circumferential portion of the guide tube facing away from the first circumferential portion.

5. A medical instrument according to claim 1, wherein the lateral cutouts comprise multiple cutouts forming a row along the guide tube longitudinal extent.

6. A medical instrument according to claim 1, wherein the lateral cutouts are configured as elongated holes or ellipses and have a longitudinal extent and a transverse extent, and wherein the longitudinal extent is at least twice the transverse extent.

7. A medical instrument according to claim 1,
wherein the lateral cutouts have a longitudinal extent which is between 10 mm and 30 mm, and/or
wherein the cutouts have a transverse extent which is between 0.15 mm and 1.0 mm.

8. A medical instrument according to claim 1,
wherein the guide tube has an outer diameter which is less than 2.2 mm, and/or
wherein the guide tube has a wall thickness which is less than 0.3 mm.

9. A medical instrument according to claim 1, wherein the cutouts are cut out with little or no post-processing by means of ultrashort pulse laser processing.

10. A medical instrument according to claim 1, wherein edges of the lateral cutouts are configured to be low in burrs or burr-free.

11. A medical instrument according to claim 1, wherein the lateral cutouts have side walls, wherein opposing side walls of a cutout enclose an outward-opening angle that is greater than 5°, and wherein the side walls are oriented radially to a longitudinal axis of the guide tube.

12. A medical instrument according to claim 1, further comprising:
a second guide tube; and
a second control element, wherein the guide tube extending parallel to the shaft is a first guide tube and the at least one control element for mechanically controlling the effector is a first control element, wherein the first guide tube and the second guide tube are arranged on the shaft as guide tubes fastened to the outside of the shaft, wherein the first guide tube accommodates the first control element and the second guide tube accommodates the second control element, and wherein the first control element and the second control element are coupled to the effector at the distal end.

13. A medical instrument according to claim 1, wherein the shaft is connected to the housing and opens into the housing, wherein the housing provides proximal access into the shaft, and wherein at least the shaft or the housing comprises at least one bore which is inclined relative to a shaft axis of the shaft and through which a housing chamber used by the controller of the effector is accessible for cleaning media.

14. A medical instrument according to claim 13, wherein the guide tube and the shaft are fluidically connected to each other via the housing chamber.

15. A medical instrument according to claim 13, wherein the guide tube opens into the housing chamber.

16. A medical instrument according to claim 13, wherein the at least one inclined bore is formed as a transverse bore through the shaft to the housing chamber, as a transverse bore oriented orthogonally to the longitudinal axis of the shaft.

17. A medical instrument according to claim 13, wherein the at least one inclined bore is formed as a connecting bore between a housing part at the proximal end of the shaft and the housing chamber, at an acute angle to the longitudinal axis, which angle opens distally.

18. A medical instrument according to claim 1, further comprising a rod, wherein the control element is coupled at a control element proximal end to the rod and the rod extends through a wall piece into the housing, and wherein a guide tube proximal end is spaced apart from the housing.

19. A method for manufacturing a guide tube of a medical instrument comprising a shaft extending between a distal end and a proximal end, a housing at the proximal end of the shaft, at least one effector at the distal end of the shaft, at least one control element for mechanically controlling the effector, and a guide tube extending parallel to the shaft, wherein the control element extends at least in portions through the guide and wherein the guide tube is provided at least in portions, along a guide tube longitudinal extent, with lateral cutouts, through which the guide tube is accessible for cleaning media, the method comprising the steps of:
providing a workpiece in a form of a semi-finished product having a thin-walled tubular body with a longitudinal axis extending between a first end and a second end;
providing an ultrashort pulse laser;
clamping the workpiece in a workpiece holder;
operating the ultrashort pulse laser to process the workpiece by sublimation;
creating a plurality of cutouts in the workpiece comprising:
generating a coordinated relative movement between the tubular body and the ultrashort pulse laser with the ultrashort pulse laser activated, comprising a translational movement along the longitudinal axis and a pivoting movement with respect to the longitudinal axis to create a cut-out section in the workpiece, which cut-out section defines each cutout, and
generating a feed movement between the tubular body and the ultrashort pulse laser with the ultrashort pulse laser deactivated, comprising a translational movement along the longitudinal axis to create the lateral cutouts that are spaced apart from one another.

20. A method according to claim 19, further comprising the step of:
removing the cut-out sections radially outward, wherein the cut-out sections have mutually inclined and opposite side walls, the angle of inclination of which points in the direction of the longitudinal axis, and/or
rotating the workpiece about the longitudinal axis until the cut-out sections can be removed with the assistance of gravity.
